(19) United States
(12) Patent Application Publication
(10) Pub. No.: US 2012/0083690 A1
(43) Pub. Date: Apr. 5, 2012

(54) SYSTEM AND METHOD FOR ELECTROMAGNETIC IMAGING AND THERAPEUTICS USING SPECIALIZED NANOPARTICLES

(76) Inventor: Serguei Y. SEMENOV, Tittensor (GB)

(21) Appl. No.: 13/252,272

(22) Filed: Oct. 4, 2011

Related U.S. Application Data
(60) Provisional application No. 61/389,638, filed on Oct. 4, 2010.

(51) Int. Cl. A61B 6/00

(52) U.S. Cl. 600/428

(57) ABSTRACT

Various systems and methods utilizing composite nanoparticles or other specialized nanoparticles in the context of electromagnetic tomography are described. A method for electromagnetic imaging includes imaging a biological tissue via an electromagnetic tomography system, recording electrical activity of the biological tissue via a biomedical electrical recording system, and correlating dielectric properties of the biological tissue with an electrical signal recorded by the biomedical electrical recording system. A method for electromagnetic imaging and therapeutics using composite nanoparticles includes imaging a biological tissue, via an electromagnetic tomography system, using a material in the composite nanoparticles, implementing a therapy, via a therapeutic application system, using a material in the composite nanoparticles, wherein the implementation is carried out at least partly on the basis of information received from the electromagnetic tomography system, assessing an effect of the therapy, and controlling further implementation of the therapy based on the assessment.

### CROSS-REFERENCE TO RELATED APPLICATION

The present application is a U.S. nonprovisional patent application of, and claims priority under 35 U.S.C. §119(e) to, U.S. provisional patent application Ser. No. 61/389,638, filed Oct. 4, 2010 and entitled “SYSTEM AND METHOD FOR ELECTROMAGNETIC IMAGING AND THERAPEUTICS USING SPECIALIZED NANOPARTICLES,” which is expressly incorporated by reference herein in its entirety.

### COPYRIGHT STATEMENT

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

### BACKGROUND OF THE PRESENT INVENTION

1. Field of the Present Invention

The present invention relates generally to the method and system for imaging and therapeutics of biological tissues using electromagnetic field (energy) and composite and other specialized nanoparticles.

2. Background

The concept of using drug loaded (or un-loaded) cancer specific magnetic nanoparticles (NPs) for therapeutics (e.g., hyperthermia) is well known (see, for example, R. Jurgons, C. Seliger, A. Hilpert, L. Trahms, S. Odenbach and C. Alexiou, “Drug loaded magnetic nanoparticles for cancer therapy,” J. Phys.: Condens. Matter 18, S2893-S2902, 2006). The use of ferroelectric nanoparticles in a hyperthermia-based cancer treatment method and system is also known, for example in U.S. Pat. No. 7,122,030. Unfortunately, these techniques suffer from drawbacks.

One shortcoming is that, in nanoparticle-mediated electromagnetic hyperthermia technologies, the distribution of nanoparticles within a biological body is unknown. Although it may be possible to use magnetic resonance imaging (MRI) to assess magnetic nanoparticle distribution, the technological efficacy (for example, localizing of small and/or time-varying concentrations of nanoparticles) and cost efficiency of such a procedure is unclear. As far as is known, there is no imaging technology available to assess biodistribution of nanoparticles, other than ones having a magnetic component or having a radiolabeling component (for example for PET or SPECT imaging). Thus, a novel technology applicable for assessing a biodistribution of nanoparticles of various compositions, but without radioactive labeling, would be desirable.

Another drawback is that the distribution of the electromagnetic field with a biological body during EM hyperthermia is also unknown, thus leading to unpredictable heating patterns. This, in turn, significantly sacrifices the selectivity and accuracy of the EM hyperthermia treating procedure.

The variable and complex nature of electromagnetic field distribution within a biological body is demonstrated via computer simulation as follows. FIG. 1 is a computer-simulated mouse model and experimental setting for use in illustrating EM field distribution. Using the model of FIG. 1, in conjunction with different EM solvers (FDTF and low-frequency) from SEMCAD (available from Schmid & Partner Engineering AG), these distributions may be simulated and analyzed. Results of one such simulation are illustrated in FIGS. 2A, 2B, 3A, 3B, 4A and 4B. FIGS. 2A and 2B are color-coded representations of the magnetic component of an EM field distribution in the form of a side view and a current loop cross-sectional (slice) view, respectively, of the mouse model; FIGS. 3A and 3B are color-coded representations of the electrical component of an EM field distribution in the form of a side view and a current loop cross-sectional (slice) view, respectively, of the mouse model; and FIGS. 4A and 4B are color-coded representations of thermogenic factor (i.e., specific absorption rate (SAR)) distribution in the form of a side view and a current loop cross-sectional (slice) view, respectively, of the mouse model.

The simulation results demonstrate the importance of knowledge of distribution of dielectric properties of tissues within a body and EM field distribution. As can be seen from the color-coded results, the EM field distribution within a biological body is very inhomogeneous, and is highly dependent on knowledge of dielectric properties of tissues and, further, on a distribution and properties of nanoparticles. Consequentially, an inhomogeneous SAR distribution, as shown in FIGS. 4A and 4B, leads to inhomogeneous generated heat patterns highlighting the previously stated need for knowledge. This is further illustrated in FIGS. 5A and 5B, which are a longitudinal cross-sectional view and a chest cross-sectional view, respectively, of an induced temperature distribution in the mouse model of FIG. 1. In FIG. 5A, the temperature palette ranges from 37 degrees C. (blue) to 40 degrees C. (yellow), while in FIG. 5B, the temperature palette ranges from 37 degrees C. (blue) to 41 degrees C. (yellow). In each case, the illustrated heat pattern is due to tissue absorption of EM energy. Use of nanoparticles with a magnetic component will provide an additional temperature increase by various mechanisms (see Rosenseweig R. E. “Heating magnetic fluid with alternating magnetic field”, J. Magn. Magn Mat., 252, 370-374, 2002). Therefore, knowledge of the distribution of dielectric properties of tissues within a body and of the distribution of nanoparticles within the body, in combination with knowledge of an EM field pattern within a body, would be very useful to the success of EM hyperthermia. Thus, an imaging modality for obtaining such knowledge directly would facilitate fast, on-line imaging of time-varying tissue properties even during an EM hyperthermia procedure.

Another shortcoming associated with current techniques is that the efficacy of an overall EM hyperthermia treatment is highly dependent on knowledge of the time-varying targeting and binding efficacy of nanoparticles, yet there is no known way to conduct an in-vivo dynamic assessment thereof. The use of an imaging modality that is capable of providing such information in dynamic, time-varying fashion would be desirable.

Still further, there is no known means for on-line monitoring of the results of an EM hyperthemia treatment in a cost efficient manner. This ability would make it possible to provide feedback, and thus correction of treatment, if needed. There are reports of using MRI for monitoring of EM hyperthermia; however, as stated previously, the technological efficacy (localizing vs time-varying changes in tissue conditions) and the cost efficiency of such a procedure is unclear.

One known technology capable of assessing dielectric properties in biological tissues is electromagnetic tomography (EMT). EMT, including microwave tomography (MWT), is a relatively recent imaging modality with great potential for biomedical applications, including a non-invasive assessment of functional and pathological conditions of biological tissues. As in any biomedical imaging, the classical EMT imaging scenario consists of cycles of measurements of complex signals, as scattered by a biologic object under study, obtained from a plurality of transmitters located at various points around the object and measured on a plurality of receivers located at various points around the object. This is illustrated in FIG. 6. As recounted elsewhere herein, the measured matrix of scattered EM signals may then be used in image reconstruction methods in order to reconstruct 2D or 3D distribution of dielectric properties of the object, i.e., to construct a 2D or 3D image of the object.

Using EMT, biological tissues are differentiated and, consequentially, can be imaged based on the differences in tissue dielectric properties. The contrast in dielectric properties between normal and abnormal tissue(s), such as, for example, malignant or infarcted tissues, thus creates the potential for EMT to be used for diagnostic purposes. In fact, the relation of dielectric properties of a tissue to its various functional and pathological conditions, such as blood and oxygen contents, ischemia and infarction malignancies, has been demonstrated. In particular, it has been shown that dielectric properties in malignant tumors and normal tissues are different in the breast, liver and lung (see W. T. Joines, Y. Zhang, C. Li, R. L. Jirtle, “The measured electrical properties of normal and malignant human tissues from 50 to 900 MHz,” Med. Phys, 31, 4, 547-550, 1994; A. J. Surowiec, S. S. Stuchly, J. R. Barr and A. Swamp, “Dielectrical properties of breast carcinoma and the surrounding tissues,” IEEE Trans. BME., 35, 4, 257-263, 1988; S. S. Chaudhary, R. K. Mishra, A. Swamp, J. M. Thomas, “Dielectric properties of normal and malignant human breast tissues at radiowave and microwave frequencies,” Indian J. Biochem., Biophys., 21, 76-79, 1984; W. T. Joines, R. J. Jirtle, M. D. Rafal, D. J. Schaefar, “Microwave power absorption differences between normal and malignant tissue,” J. Radiation Oncology Biol. Phys., 6, 681-687, 1980; M Lazebnik, L. McCartney, D. Popovic, C. Watkins, M. J. Lindstrom, J. Harter, S. Sewall, A. Magliocco, J. H. Booske, M. Okoniewski and S. C. Hagness, “A large-scale study of the ultrawideband microwave dielectric properties of normal breast tissue obtained from reduction surgeries,” Phys. Med. Biol, 52, 2637-2656, 2007; M. Lazebnik, D. Popovic, L. McCartney, C. B. Watkins, M. J. Lindstrom, J. Harter, S. Sewall, T. Ogilvie, A. Magliocco, T. M. Breslin, W. Temple, D. Mew, J. H. Booske, M. Okoniewski and S. C. Hagness, “A large-scale study of the ultrawideband microwave dielectric properties of normal, benign and malignant breast tissues obtained from cancer surgeries,” Phys. Med. Biol, 52, 6093-6115, 2007; S. R. Smith, K. R. Foster, G. L. Wolf, “Dielectric properties of VX-2 carcinoma versus normal liver tissue,” IEEE Trans. BME, 33, 5 522-524, 1986; A. P. O'Rourke, M. Lazebnik, J. M. Bertram, M. C. Converse, S. C. Hagness, J. G. Webster and D. M. Mahvi, “Dielectric properties of human normal, malignant and cirrhotic liver tissue: in vivo and ex vivo measurements from 0.5 to 20 GHz using a precision open-ended coaxial probe,” Phys. Med. Biol, 52, 4707-4719, 2007; T. Marimoto, S. Kimura, Y. Konishi, K. Komaki, T. Ugama, Y. Monden, Y. Kinochi, T. Iritana, “A study of electrical bio-impedance of tumors,” J. of Investigative Surgery, 6, 25-32, 1993), and that dielectric properties of myocardial tissue have strong dependence from coronary blood flow, myocardial hypoxia, acute ischemia and chronic infarction (see S. Y. Semenov, R. H. Svenson and G. P. Tatsis, “Microwave spectroscopy of myocardial ischemia and infarction. 1. Experimental study,” Annals of Biomed. Eng. 28, 48-54, 2000; S. Y. Semenov, R. H. Svenson, V. G. Posukh, A. G. Nazarov, Y. E. Sizov, J. Kassel and G. P. Tatsis, “Dielectric spectroscopy of canine myocardium during ischemia and hypoxia at frequency spectrum from 100 KHz to 6 GHz,” IEEE Trans. MI 21, 703-707, 2002).

EMT technology, such as that disclosed in U.S. Pat. No. 6,490,471; U.S. Pat. No. 6,332,087; U.S. Pat. No. 6,026,173; and U.S. Pat. No. 5,715,819, has existed for many years. Somewhat more recently, it has been suggested that ferroelectric nanoparticles may be utilized for contrast enhancement of EMT. For example, U.S. Pat. No. 7,239,731 suggests the use of ferroelectrics, having dielectric properties that are a function of an electrical field generated by biological excited tissue, as one possible sensitive material (solution) to be injected into a biological material or in a circulation system in a method for non-destructive detection and mapping of electrical excitation of biological tissues with the help of electromagnetic field tomography and spectroscopy (see also S. Semenov, N. Pham and S. Egot-Lemaire, “Ferroelectric Nanoparticles for Contrast Enhancement Microwave Tomography: Feasibility Assessment for Detection of Lung Cancer,” World Congress on Medical Physics and Biomedical Engineering, Munich, Germany, Sep. 7-12, 2009). Ferroelectrics, having high values of dielectric properties as compared with biological tissues, present interesting enhancement potentials. The efficiency of using such an approach has been at least partially demonstrated using computer simulation and a 2D human chest model. In this regard, FIGS. 7A and 7B are reconstructed MWT images for ∈′ and ∈″, respectively, of a 2D chest model with two simulated cancer areas. In each image, the upper cancer area is shown with 15% dielectric contrast at [X,Y]=(−9.0 cm, 3.0 cm) and Radius=10 mm, while the lower cancer area is shown with ferroelectric nanoparticles (with 0.5 volume fraction) enhanced contrast at [X,Y]=(−5.0 cm, 8.25 cm) and Radius=5 mm. The frequency in the simulation was 1 GHz.

In view of the foregoing discussion, it will be appreciated that certain nanoparticles, such as magnetic nanoparticles, are or may be an effective tool in various treatments and therapeutics, while other nanoparticles, particularly including ferroelectric nanoparticles, are useful in imaging modalities, particularly EMT. However, a need exists for a cost-efficient technology whereby the benefits achieved using these different nanoparticles are combined together, thereby enhancing both and providing heretofore unrealized benefits.

### SUMMARY OF THE PRESENT INVENTION

According to at least one aspect, the present invention includes a method and system for electromagnetic imaging and therapeutics enhanced by using composite nanoparticles (NPs). Among other benefits or advantages, such method and system not only improve both imaging and therapeutics components, but add a new dimension to both components. This is a non-invasive control of nanoparticles targeting efficiency and, consequentially, a novel way of selective therapeutics for nanoparticles bound with targeting cells only.

Broadly defined, the present invention according to one aspect is a system as shown and described.

Broadly defined, the present invention according to another aspect is a method as shown and described.

Broadly defined, the present invention according to another aspect is a system for assessing binding efficiency of composite nanoparticles with biological cells as shown and described.

Broadly defined, the present invention according to another aspect is a method for assessing binding efficiency of composite nanoparticles with biological cells as shown and described.

Broadly defined, the present invention according to another aspect is a system for electromagnetic imaging using nanoparticles as shown and described.

In a feature of this aspect, electromagnetic imaging is carried out via an electromagnetic tomography system.

In further features, the nanoparticles are composite nanoparticles; the nanoparticles include ferroelectric and magnetic components; and/or the nanoparticles include ferroelectric and metal components, wherein the metal component includes gold and/or wherein the metal component includes copper; and/or the nanoparticles include magnetic and metal components, wherein the metal component includes gold and/or copper.

In still further features, the nanoparticles include a ferroelectric component; the nanoparticles include a magnetic component; and/or the nanoparticles include a metal component, wherein the metal component includes gold and/or copper.

Broadly defined, the present invention according to another aspect is a method of electromagnetic imaging using nanoparticles as shown and described.

Broadly defined, the present invention according to another aspect is a method of electromagnetic imaging using nanoparticles as shown and described.

In a feature of this aspect, imaging is carried out via electromagnetic tomography.

In further features, the nanoparticles are composite nanoparticles; the nanoparticles include ferroelectric and magnetic components; and/or the nanoparticles include ferroelectric and metal components, wherein the metal component includes gold and/or wherein the metal component includes copper; and/or the nanoparticles include magnetic and metal components, wherein the metal component includes gold and/or copper.

In still further features, the nanoparticles include a ferroelectric component; the nanoparticles include a magnetic component; and/or the nanoparticles include a metal component, wherein the metal component includes gold and/or copper.

Broadly defined, the present invention according to another aspect is a system for electromagnetic imaging and therapeutics using composite nanoparticles as shown and described.

Broadly defined, the present invention according to another aspect is a method of electromagnetic imaging and therapeutics using composite nanoparticles as shown and described.

Broadly defined, the present invention according to another aspect is a system for electromagnetic imaging using nanoparticles, including: an electromagnetic tomography system adapted to image a biological tissue; a biomedical electrical recording system adapted to record electrical activity of the biological tissue; and a control integration system adapted to correlate dielectric properties of the biological tissue with an electrical signal recorded by the biomedical electrical recording system.

In features of this aspect, the biomedical electrical recording system is an ECG system; the biomedical electrical recording system is an EEG system; the biomedical electrical recording system is an EMG system; and/or the biomedical electrical recording system is an EvP system.

In another feature of this aspect, electromagnetic tomography system images the biological tissue via nanoparticles, introduced into the biological tissue, that have dielectric properties that are a function of electrical field, generated by biological excited tissue. In further features, the nanoparticles are ferroelectric nanoparticles; and the nanoparticles are introduced into the biological tissue via injection and/or the nanoparticles are introduced into the biological tissue via circulation system.

In another feature of this aspect, the control integration system is adapted to correlate a reconstructed distribution (image) of dielectric properties of the biological tissue at each geometrical point (x,y,z) within the biological tissue.

Broadly defined, the present invention according to another aspect is a method for electromagnetic imaging using nanoparticles, including: imaging a biological tissue via an electromagnetic tomography system; recording electrical activity of the biological tissue via a biomedical electrical recording system; and correlating dielectric properties of the biological tissue with an electrical signal recorded by the biomedical electrical recording system.

In features of this aspect, the step of recording electrical activity of the biological tissue comprises recording an ECG signal via an ECG system; the step of recording electrical activity of the biological tissue comprises recording an EEG signal via an EEG system; the step of recording electrical activity of the biological tissue comprises recording an EMG signal via an EMG system; and/or the step of recording electrical activity of the biological tissue comprises recording an EvP signal via an EvP system.

In another feature of this aspect, imaging the biological tissue via an electromagnetic tomography system includes introducing nanoparticles into the biological tissue, wherein the nanoparticles have dielectric properties that are a function of electrical field generated by biological excited tissue. In further features, introducing nanoparticles includes introducing ferroelectric nanoparticles; and the nanoparticles are introduced into the biological tissue via injection and/or the nanoparticles are introduced into the biological tissue via circulation system.

In another feature of this aspect, the step of correlating dielectric properties of the biological tissue with an electrical signal recorded by the biomedical electrical recording system is carried out by a control integration system.

In another feature of this aspect, the step of correlating dielectric properties of the biological tissue with an electrical signal recorded by the biomedical electrical recording system includes correlating a reconstructed distribution (image) of dielectric properties of the biological tissue at each geometrical point (x,y,z) within the biological tissue.

Broadly defined, the present invention according to another aspect is a system for electromagnetic imaging and therapeutics using composite nanoparticles, including: an electromagnetic tomography system adapted to image a biological tissue via a first material in the composite nanoparticles; a therapeutic application system adapted to implement a therapy, via a second material in the composite nanoparticles, at least partly on the basis of information received from the electromagnetic tomography system; and a control system adapted to assess an effect of the therapy and to control further implementation of the therapy based on the assessment.

In a feature of this aspect, the electromagnetic tomography system is adapted to image the biological tissue via a first material in the composite nanoparticles, and the therapeutic application system is adapted to implement the therapy via a second material in the composite nanoparticles. In further features, the first material in the composite nanoparticles includes a ferroelectric material; the second material in the composite nanoparticles includes a magnetic material; the therapy utilizes an electrical mechanism and/or the therapy utilizes a thermogenic mechanism; and the therapeutic application system includes antennas disposed at points around the biological tissue and/or the therapeutic application system includes one or more coil arranged around the biological tissue.

In another feature of this aspect, the nanoparticle material via which the electromagnetic tomography system is adapted to image the biological tissue is the same nanoparticle material via which the therapeutic application system is adapted to implement the therapy.

Broadly defined, the present invention according to another aspect is a method for electromagnetic imaging and therapeutics using composite nanoparticles, including: imaging a biological tissue, via an electromagnetic tomography system, using a first material in the composite nanoparticles; implementing a therapy, via a therapeutic application system, using a second material in the composite nanoparticles, wherein the implementation is carried out at least partly on the basis of information received from the electromagnetic tomography system; assessing an effect of the therapy; and controlling further implementation of the therapy based on the assessment.

In a feature of this aspect, the step of imaging the biological tissue uses a first material in the composite nanoparticles, and the step of implementing the therapy uses a second material in the composite nanoparticles. In further features, the first material in the composite nanoparticles includes a ferroelectric material; the second material in the composite nanoparticles includes a magnetic material; implementing the therapy includes utilizing an electrical mechanism and/or implementing the therapy includes utilizing a thermogenic mechanism; and implementing the therapy includes disrupting or opening pores in the cellular membrane of targeted cells and/or implementing the therapy includes disrupting or opening pores in the shell of the nanoparticles to release a drug therefrom.

In another feature of this aspect, the step of imaging the biological tissue and the step of implementing the therapy use the same material in the composite nanoparticles.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

Further features, embodiments, and advantages of the present invention will become apparent from the following detailed description with reference to the drawings, wherein:

FIG. 1 is a computer-simulated mouse model and experimental setting for use in illustrating nanoparticle and EM field distribution;

FIGS. 2A and 2B are color-coded representations of a magnetic component of EM field distribution in the form of a side view and a current loop cross-sectional (slice) view, respectively, of the mouse model of FIG. 1;

FIGS. 3A and 3B are color-coded representations of an electrical component of EM field distribution in the form of a side view and a current loop cross-sectional (slice) view, respectively, of the mouse model of FIG. 1;

FIGS. 4A and 4B are color-coded representations of thermogenic factor (i.e., SAR) distribution in the form of a side view and a current loop cross-sectional (slice) view, respectively, of the mouse model of FIG. 1;

FIGS. 5A and 5B, which are a longitudinal cross-sectional view and a chest cross-sectional view, respectively, of an induced temperature distribution in the mouse model of FIG. 1;

FIG. 6 is a graphical illustration of the principle of electromagnetic tomography (EMT);

FIGS. 7A and 7B are reconstructed MWT images for ∈′ and ∈″, respectively, of a 2D chest model with two simulated cancer areas;

FIG. 8 is a high-level block diagram of a system for electromagnetic imaging and therapeutics using composite nanoparticles in accordance with one or more preferred embodiments of the present invention;

FIG. 9 is a schematic view of an EM field tomographic spectroscopic system suitable for use as the EMT system of FIG. 8;

FIG. 10 is a block diagram of one of the N EM field clusters of FIG. 9, wherein the cluster is in its source state;

FIG. 11 is a block diagram of one of the M source-detector modules of FIG. 10;

FIG. 12 is a block diagram of the R-channel module of FIG. 10;

FIG. 13 is a block diagram of one of the IF detector clusters of FIG. 9;

FIG. 14 is a block diagram of the control system for the EM field clusters and IF detector clusters of FIG. 9;

FIG. 15 is a block diagram illustrating the integration of the control system of FIG. 13 with the system of FIG. 9;

FIG. 16 is a block diagram of the EM field source-detector cluster of FIG. 10, wherein the cluster is in its detector state;

FIG. 17 is a schematic diagram illustrating the acquisition of raw data via both the EMT system and the biomedical electrical recording system of FIG. 8;

FIG. 18 is a schematic diagram illustrating a process of imaging and determination of correlation of the data from the EMT system with the data from the biomedical electrical recording system as carried out by the control integration system, all in accordance with one or more preferred embodiments of the present invention;

FIGS. 19A and 19B are graphical illustrations of computer simulation results of the radial distribution of electric field E and thermogenic factor (σ*E*E), at various frequencies, in and across the boundary of an exemplary nanoparticle;

FIGS. 20A and 20B are schematic diagrams illustrating the use of an EMT imaging protocol in conjunction with a therapeutic protocol in accordance with one or more preferred embodiments of the present invention;

FIG. 21 is an illustrative computer-simulated human head model and experimental setting for use in illustrating the protocols of FIGS. 20A and 20B; and

FIG. 22 is a tabular representation of the functionality carried out by the antennas and coil during the imaging protocol and the therapeutic protocol of FIGS. 20A and 20B, respectively.

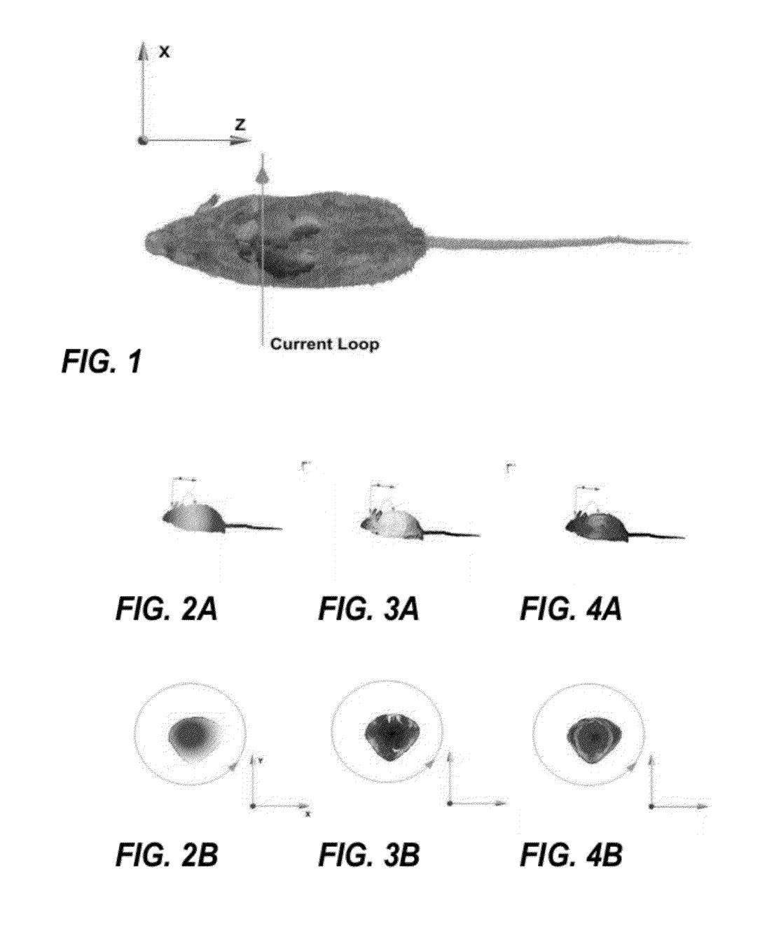

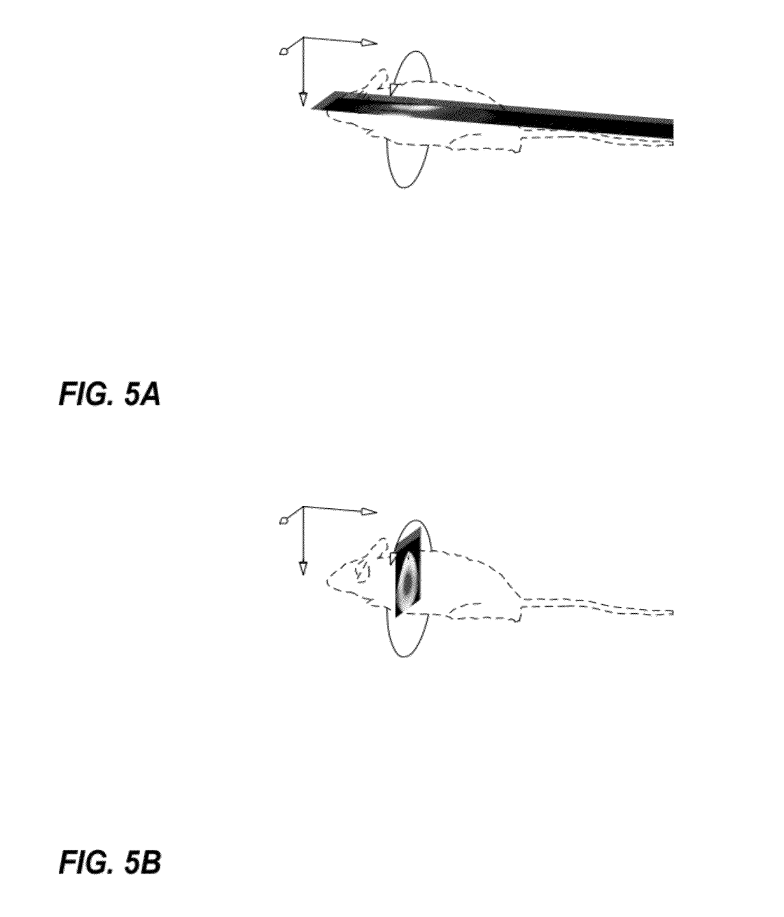

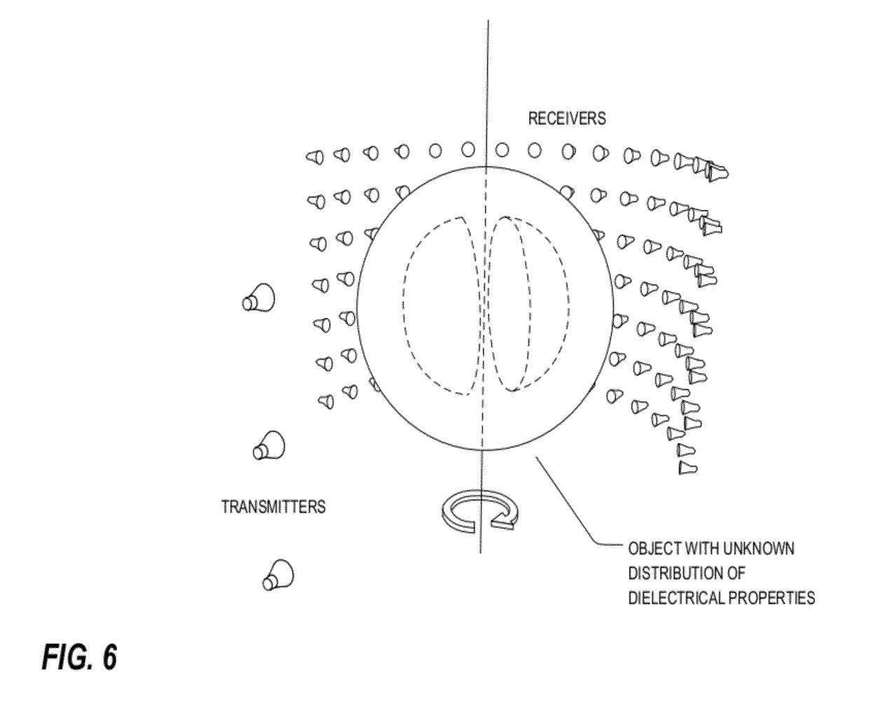

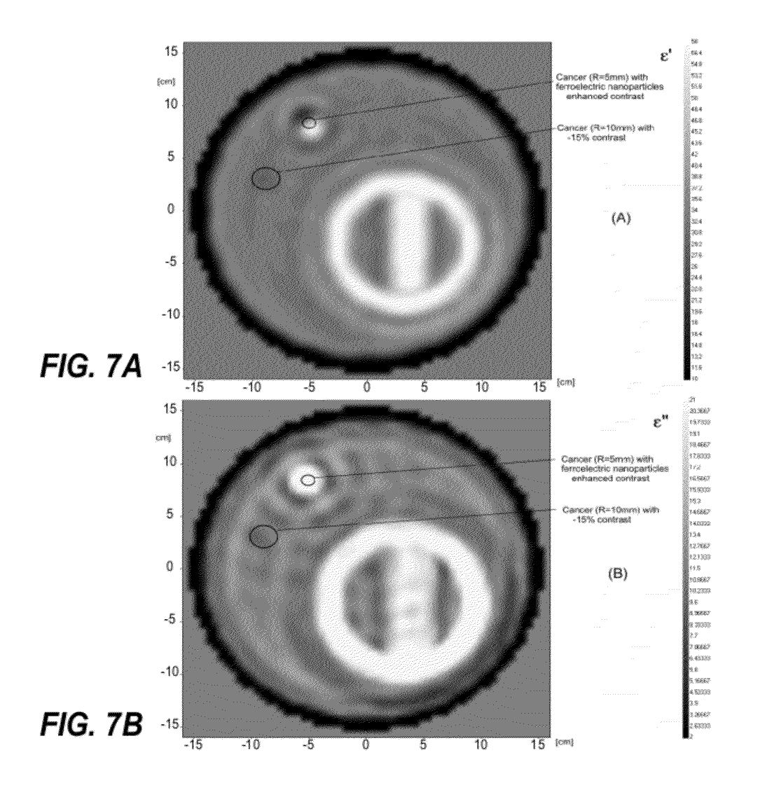

### DETAILED DESCRIPTION

As a preliminary matter, it will readily be understood by one having ordinary skill in the relevant art (“Ordinary Artisan”) that the present invention has broad utility and application. Furthermore, any embodiment discussed and identified as being “preferred” is considered to be part of a best mode contemplated for carrying out the present invention. Other embodiments also may be discussed for additional illustrative purposes in providing a full and enabling disclosure of the present invention. As should be understood, any embodiment may incorporate only one or a plurality of the above-disclosed aspects of the invention and may further incorporate only one or a plurality of the above-disclosed features. Moreover, many embodiments, such as adaptations, variations, modifications, and equivalent arrangements, will be implicitly disclosed by the embodiments described herein and fall within the scope of the present invention.

Accordingly, while the present invention is described herein in detail in relation to one or more embodiments, it is to be understood that this disclosure is illustrative and exemplary of the present invention, and is made merely for the purposes of providing a full and enabling disclosure of the present invention. The detailed disclosure herein of one or more embodiments is not intended, nor is to be construed, to limit the scope of patent protection afforded the present invention, which scope is to be defined by the claims and the equivalents thereof. It is not intended that the scope of patent protection afforded the present invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection afforded the present invention is to be defined by the appended claims rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

Regarding applicability of 35 U.S.C. §112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at least one of the items,” but does not exclude a plurality of items of the list. Thus, reference to “a picnic basket having cheese or crackers” describes “a picnic basket having cheese without crackers,” “a picnic basket having crackers without cheese,” and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

Referring now to the drawings, in which like numerals represent like components throughout the several views, the preferred embodiments of the present invention are next described. The following description of one or more preferred embodiment(s) is merely exemplary in nature and is in no way intended to limit the invention, its application, or uses.

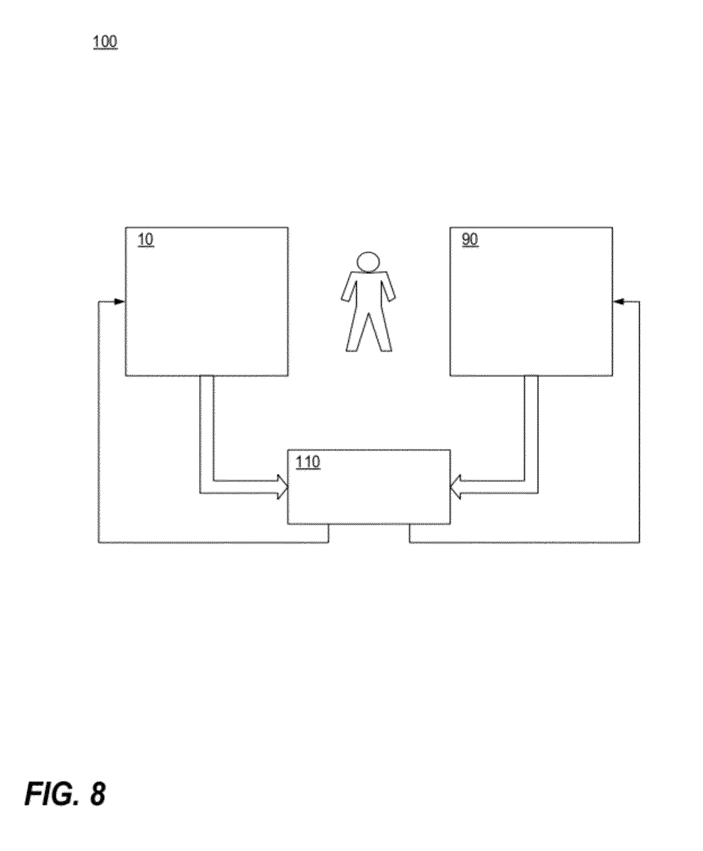

FIG. 8 is a high-level block diagram of a system 100 for electromagnetic imaging and therapeutics using composite nanoparticles in accordance with one or more preferred embodiments of the present invention. As shown therein, the system 100 includes an EMT system 10, a biomedical electrical recording system 90, and a control integration system 110. The biomedical electrical recording system 90 may be any conventional or newly-developed system for recording any electrical activity of any biological tissue, including an ECG system for recording cardiac electrical excitation, an EEG system for recording brain electrical excitation, an EMG system for recording skeletal muscle electrical excitation, an EvP system for recording muscle evoked potentials, or the like. The control integration system 110 includes functional components for integrating data from the EMT system 10 with data from the biomedical electrical recording system 90 as further described below.

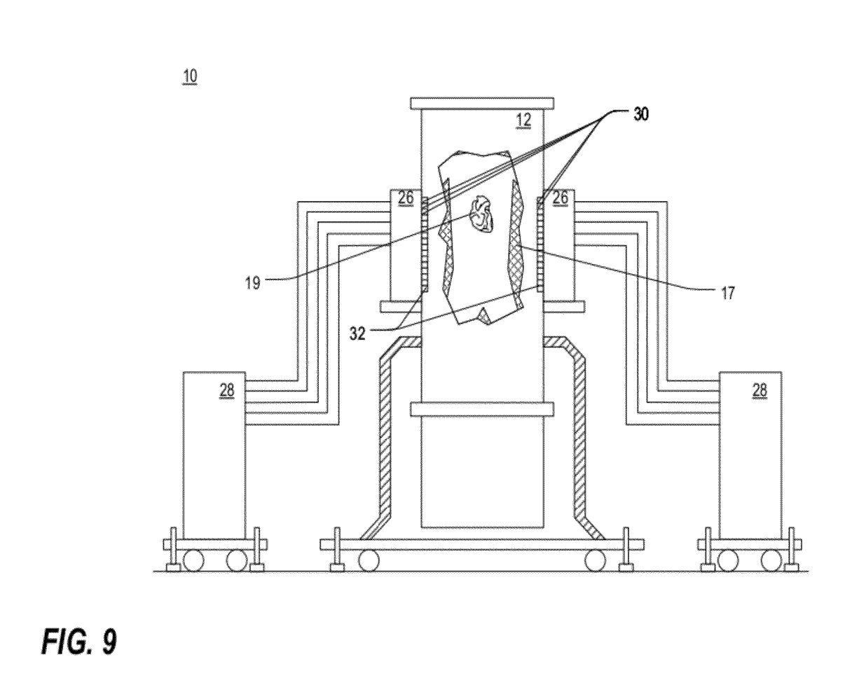

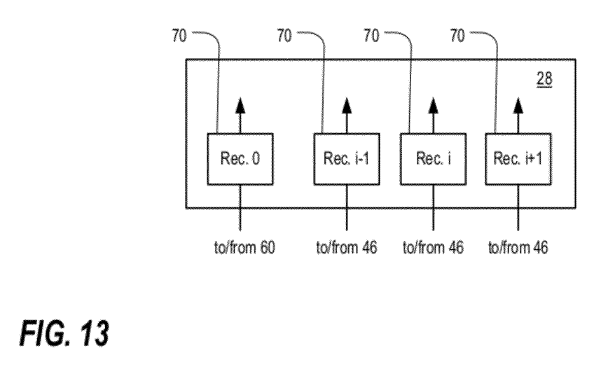

FIG. 9 is a schematic view of an EM field tomographic spectroscopic system suitable for use as the EMT system 10 of FIG. 8. In particular, the system 10 of FIG. 9 carries out functional imaging of biological tissues. The system 10 might also be used for a non-invasive mapping of electrical excitation of biological tissues 19 using a sensitive (contrast) material (solution or nanoparticles), injected into the biological tissue 19 or into the circulation system, characterized by having dielectric properties that are a function of electrical field, generated by biological excited tissue 19. As illustrated in FIG. 9, the illustrative system 10 includes a working or imaging chamber 12, a plurality of “EM field source-detector” clusters 26, an equal number of intermediate frequency (“IF”) detector clusters 28, and a control system (not shown in FIG. 9, but illustrated in block diagram form in FIG. 13). Although for simplicity only two EM field source-detector clusters 26 and two IF detector clusters 28 are shown in FIG. 9, it should be clear that a much larger number of each, sometimes denoted herein by N, may (and in at least most cases preferably should) be used.

The imaging chamber 12 may be a watertight vessel of sufficient size to accommodate a human body or one or more portions of a human body. For example, the imaging chamber 12 may be i) a helmet-like imaging chamber to image brain disorders (for example acute and chronic stroke), ii) a cylindrical type chamber for extremities imaging, or iii) a specifically shaped imaging chamber for detection of breast cancer. Therefore an imaging chamber may have different shapes and sizes, the selection of which would be readily apparent to one of ordinary skill in the art. In at least one embodiment, the imaging chamber 12 and its EM field clusters 26, as well as the IF detector clusters 28, may be mounted on carts in order to permit the respective components to be moved if necessary, and the carts may then be locked in place to provide stability.

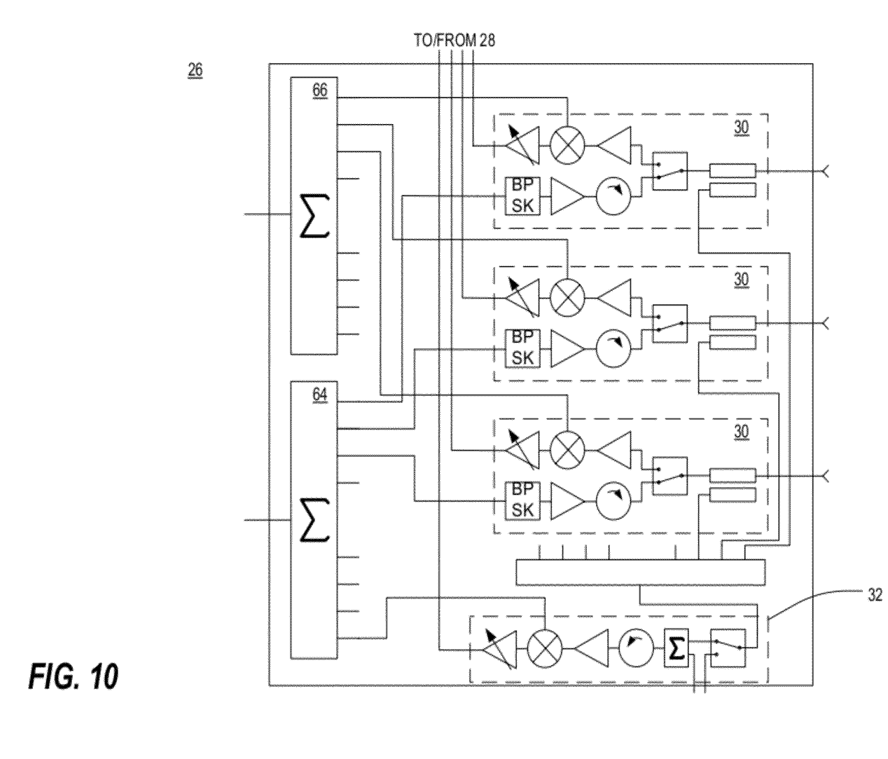

FIG. 10 is a block diagram of one of the N EM field clusters 26 of FIG. 9, wherein the cluster 26 is in its source state. Each EM field cluster 26 is a main operation unit that may function as an electromagnetic field generator (i.e., an electromagnetic source) or as an electromagnetic field detector. Each cluster 26 has a plurality of source-detector modules 30, one reference channel (“R-channel”) module 32 and a pair of distribution blocks 64,66, as well as at least two precision attenuators. The number of source-detector modules 30 (three being shown here) in each EM field cluster 26 may sometimes be denoted herein by M. In general, the more source-detector modules 30 that are used, the greater the precision of the system 10. However, because of the large amounts of data that are created, it may be preferable in at least some embodiments to use between 500 and 1500 source-detector modules 30, with an optimum number near 1000, organized into N EM field clusters 26, with the value of N selected based generally on manufacturability and convenience.

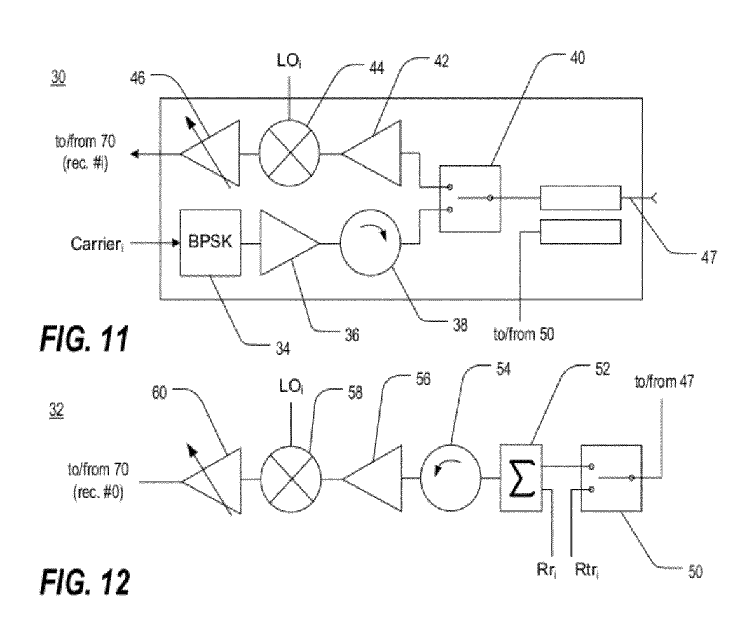

FIG. 11 is a block diagram of one of the M source-detector modules 30 of FIG. 10. Each source-detector 30 includes a BPSK modulator 34, a power amplifier 36, a direct uncoupler 38, a switch 40, a low noise amplifier (“LNA”) 42, a mixer 44, a programmable gain amplifier (“PGA”) 46 and an antenna 48. The switch 40 functions to connect the antenna 48 into the system 10 as an EM source or as an EM detector. When connected as a source (i.e., when the switch 40 is in the lower of the two positions shown in FIG. 11), an input signal provided by one of the distribution blocks 64 (as shown in FIG. 10) is modulated by the BPSK modulator 34, amplified by the amplifier 36 and uncoupled by the direct uncoupler 38 before passing through the switch 40 to the antenna 48. On the other hand, when connected as a detector (i.e., when the switch 40 is in the upper of the two positions shown in FIG. 11), the signals received by the antenna 48 pass through the switch 40 to the LNA 42 where they are amplified and then mixed with a reference signal provided by the second distribution block 66 (as shown in FIG. 10) and then amplified again by the PGA 46.

FIG. 12 is a block diagram of the R-channel module 32 of FIG. 10. As described previously, there are preferably a plurality (M) of source-detector modules 30 in each EM field cluster 26 but only a single R-channel module 32. The R-channel module 32 includes a switch 50, an adder 52, a direct uncoupler 54, an LNA 56, a mixer 58 and a PGA 60. The switch 50 controls whether the R-channel module 32 is in its source state or its detector state. When the R-channel module 32 is in its source state (i.e., when the switch 40 is in the upper of the two positions shown in FIG. 12), output signals from the source-detector modules 30 are passed through the adder 52 and the direct uncoupler 54 and are amplified by the LNA 56 before being mixed with a reference signal and amplified again by the PGA 60. On the other hand, when the R-channel module 32 is in its detector state (i.e., when the switch 40 is in the lower of the two positions shown in FIG. 12), a reference signal is passed straight through to the source-detector modules 30 where it is coupled with the signals received by the respective antennae 48.

FIG. 13 is a block diagram of one of the IF detector clusters 28 of FIG. 9. Each IF detector cluster 28 includes a family of M+1 digital correlation detectors 70 for M test signals (one from each of the source-detector modules 30 in a corresponding EM field cluster 26) and one reference channel signal. These digital detectors 70 allow for the informative/working bandwidth of the signal to be selectively passed while restricting other artifacts. Each IF detector cluster 28 also includes a cluster manager, a bus, and a power supply.

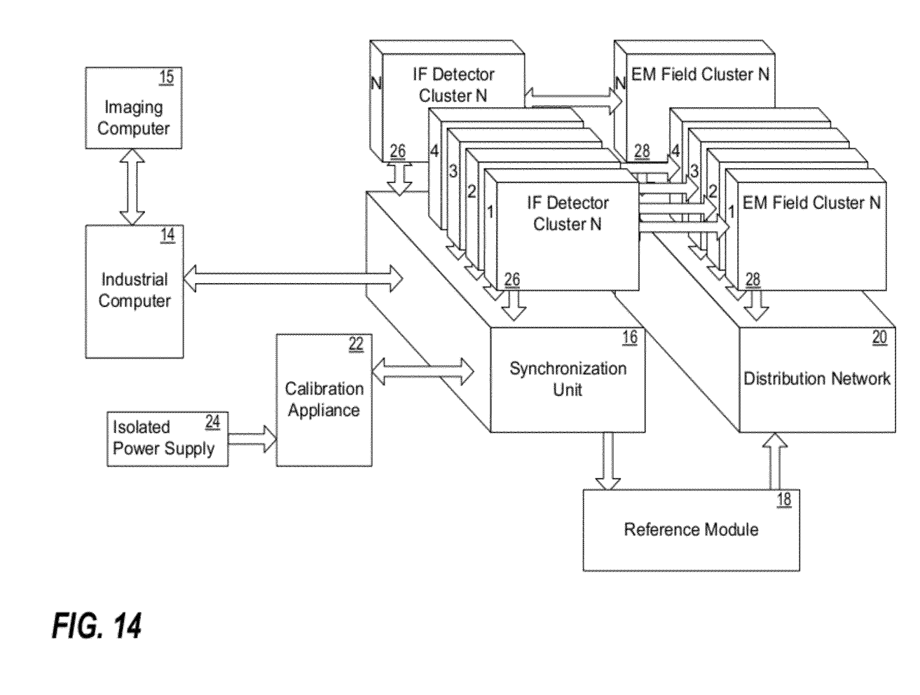

FIG. 14 is a block diagram of the control system for the EM field clusters 26 and IF detector clusters 28 of FIG. 9. The control system includes a control computer 14, an imaging computer 15, a synchronization unit 16, a reference module 18, a distribution network 20, a calibration appliance 22 and a power supply 24. The control computer 14 controls the overall system function, data acquisition, system tuning and calibration and transforms all raw data to the imaging computer 15 for further data inversion and imaging. The control computer 14 may be a conventional personal computer, such as an Intel-based advanced-version PC, with an RS-488.2 port and appropriate software to control the system 10. The synchronization unit 16 is a module that includes a system manager and a system hub. Together, they provide data exchange with the control computer 14 (preferably via a USB 2.0 or Firewire link) and the control managers of the various clusters 26,28, and also provide synchronization of system operations.

The reference module 18 includes two generators, one or more thermostats for temperature stabilization of the function of the reference channels, a BPSK modulator for phase-modulation, power dividers, attenuators and the like. The two generators are precision generators that generate stable CW signals: Carrier_{ref} and LO_{ref}. These generators are controlled and tuned by the control computer 14 through an interface. The distribution network 20 is a commutation unit for receiving the carrier and local oscillator reference signals (Carrier_{ref} and LO_{ref}) and the Rr and Rtr reference signals (Rr_{ref} and Rtr_{ref}) from the reference module 18 and distributing them to each of the EM field clusters 28.

The calibration appliance 22 is used for calibration and fine-tuning of the system 10. The calibration appliance 22 includes a calibration source, one or more (preferably two) calibration antennae, precision drives and one or more (preferably three) calibrated phantoms. Calibration antennae and phantoms may be precisely positioned at any point inside the imaging chamber 12 with the help of precision positioning drivers. The isolated power supply 24 provides stable power for the system. One power supply suitable for use with the present invention is a 190/380 3-phase, 10 kVA AC network power supply. Of course, the exact requirements for the power supply 24 may depend upon the power system specifications of the country in which the system 10 is to be operated.

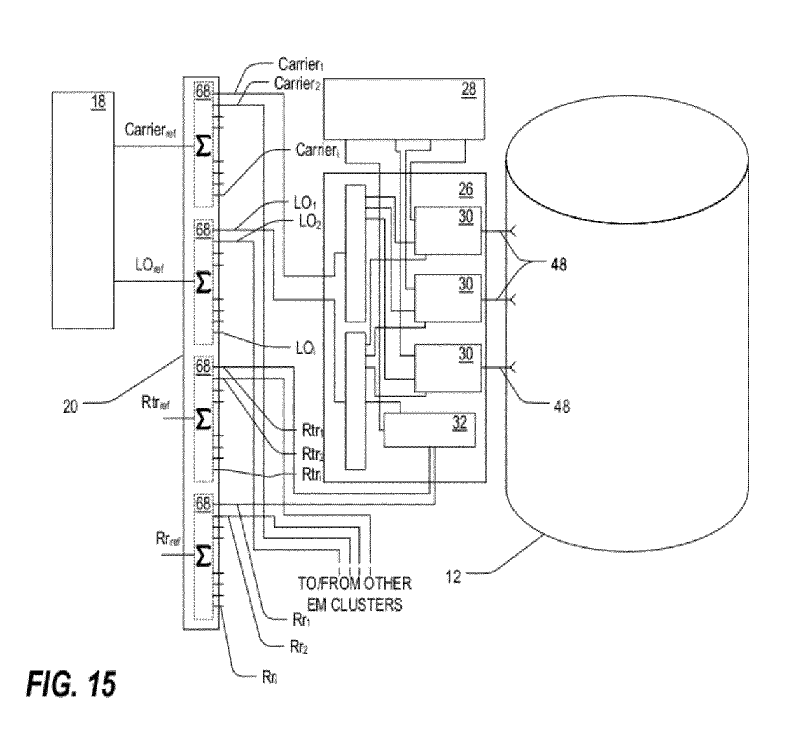

FIG. 15 is a block diagram illustrating the integration of the control system of FIG. 13 with the system 10 of FIG. 9. Each EM field cluster 26 is disposed adjacent the imaging chamber 12 such that its antennae 48 are located on or near the surface of the chamber 12. The outputs of the source-detector modules 30 and the R-channel module 32 of each EM field cluster 26 are connected to a corresponding IF detector cluster 28, and each IF detector cluster 28 is connected to both the corresponding EM field cluster 26 and the synchronization unit 16. The inputs of each EM field cluster 26 are connected to the distribution network 20. The distribution network 20 includes at least four distribution blocks 68, which may be 34-channel power dividers, and a system bus for distributing the various reference signals (Carrier_{ref}, LO_{ref}, Rr_{ref} and Rtr_{ref}) to the EM field clusters 26. As illustrated in FIG. 14, one set of the four signals is provided to each EM field cluster 26. These signals are denoted Carrierᵢ, LOᵢ, Rrᵢ and Rtrᵢ, where the first EM field cluster 26 receives Carrier₁, LO₁, Rr₁ and Rtr₁, the second EM field cluster 26 (not separately illustrated) receives Carrier₂, LO₂, Rtr₂ and Rtr₂, and so forth. Finally, as described previously, Carrier_{ref} and LO_{ref} are provided to the distribution network 20 by the reference module 18.

The EMT system 10 described previously may be used to assess the nanoparticles' binding efficiency at each 3D point within a domain of interest (i.e., a biological object) using composite or multi-component nanoparticles. In particular, the composite nanoparticles may include a component whose dielectric properties are a function of the electrical field generated by the biological excited tissue 19 itself, such as a ferroelectric nanoparticle. One suitable ferroelectric nanoparticle is barium modified strontium titanium oxide, of different grain sizes, which in some embodiments may include spheres, ellipsoids, cylinders, and/or the like. Specific functionalized nanoparticles might also include a magnetic nano-component (such as magnetite or cobalt-ferrite) biologically compatible shells with specific biological targeting and a desired delivery drug. The materials of NPs may also include other potentiometric components, for example potentiometric dyes, such as merocyanine, rhodamine, cyanine, oxonol and naphthyl styryl, and/or selected potentiometric liquid crystals, such as MBBA, 7CB.

A basic principle of operation of at least some embodiments of the system 100 of the present invention is as follows. Due to the presence of the ferroelectric component (or any other membrane potential-sensitive component, including those listed above) in the composite nanoparticles, if a nanoparticle binds with the cellular membrane of an electrically excitable cell, then its dielectric properties depend on the phase of cellular electrical excitation, since the dielectric properties of the nanoparticles depend on local electrical potential. Therefore, if during the execution of an EMT dynamic imaging protocol there is, for the duration of at least one excitation cycle with sufficiently timely resolution, a component of a reconstructed image (i.e., a reconstructed 2D or 3D distribution of dielectric properties ∈(x,y,z)), at a particular point (xₐ,yₐ,zₐ), that correlates with an electrical excitation that is measured independently by the biomedical electrical recording system 90, then it can be concluded that nanoparticles do bind with cellular membranes at this point (xₐ,yₐ,zₐ). This means that an EMT dynamic imaging protocol will lead to a 2D or 3D matrix of correlation coefficients corr(x,y,z) corresponding to the domain of interest. The degree of binding at any point (x,y,z) in the entire domain of interest is directly proportional to the value of corr(x,y,z).

In use, the imaging chamber 12 may be filled with one of a variety of solutions or gels 17 selected to match and provide biological compatibility with a biological tissue object 19 to be studied. Suitable solutions 17 may include, but are not limited to, water, salt solutions, sugar solutions, fatty emulsions, alcohol-salt mixed solutions and the like; these solutions may also be used as gel components. The object 19 to be studied is injected with a sensitive material (solution) (or distributed in the object 19 via the circulation system) that includes the desired composite or multi-component nanoparticles.

Before placing the object under study 19 in the imaging chamber 12, a system calibration and test procedure is preferably conducted. The EMT system 10 is calibrated, and EM fields in a so called “EMPTY” imaging domain are measured. “EMPTY” EM fields are the fields measured within an imaging domain when the domain is filled in with matching solution but there is no object of interest 19 inside the domain. Then, the measured “EMPTY” EM fields from all possible Transmitters/Receivers (Tx/Rx) combinations are compared with “standard EM field matrix.” The “standard EM field matrix” is obtained previously by comparing results of computer simulations with a series of measurements at the same “EMPTY” imaging domain filled in with various matching solutions of different dielectric properties. The dielectric properties of the matching solution that is being used is independently measured by means of a well-known contact dielectric probe method. Preferably, system stability tests are later run to ensure that the system on-time performance is in a satisfactory range.

The object under study 19 is positioned inside the imaging chamber 12 and both the EMT system 10 and the biomedical electrical recording system 90 are activated. During operation of the EMT system 10, each Carrierᵢ signal from the signal generator in the reference module 18 is provided to a source-detector module 30, operating in its source state as shown in FIG. 10, where it is modulated using phase-shift modulation (in case of phase characterization) by pseudo-random code in order to distinguish each transmitting antenna 48 or source from the other antennae/sources 48, which are transmitting simultaneously. As described previously, the resultant signal is next amplified before passing through the direct uncoupler 38 to the appropriate source antenna 48. As a result, an incident EM field (“E_{inc}”), corresponding to the respective antenna 48 or channel, is formed in the vicinity of the object 19 under study. In addition, part of the signal creating the E_{inc} field is uncoupled and passed to a receiver in the R-channel module 32 (one for each EM field cluster 26). In the R-channel module 32 this signal is mixed with a reference signal Rrᵢ. By subsequently comparing the resultant output with a known signal, E_{inc} may thus be determined precisely as described below.

### Operation of EMT System

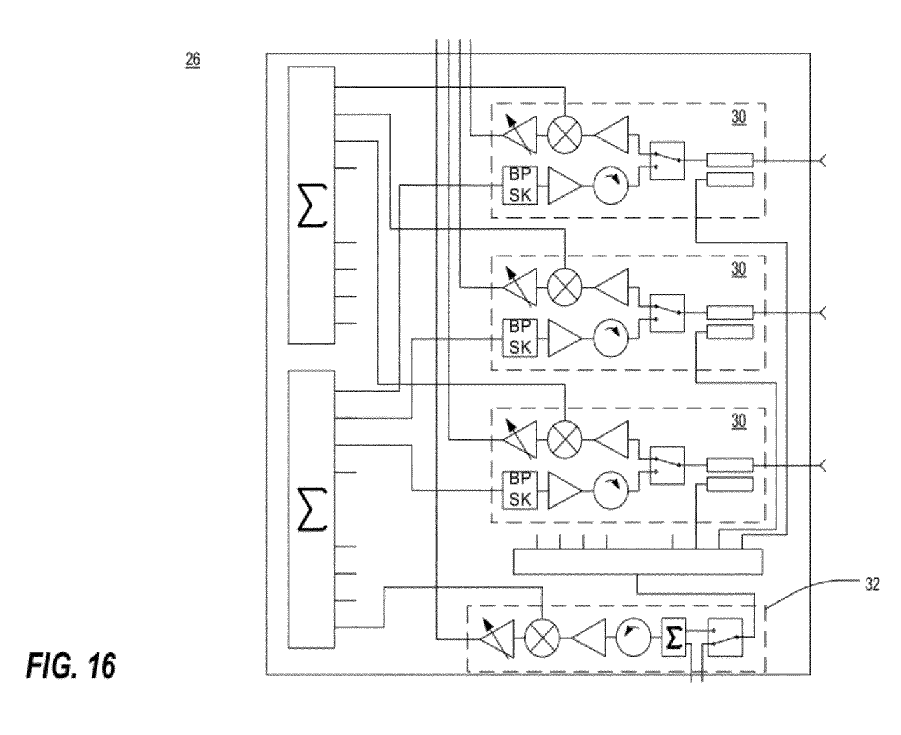

After interacting with the object 19 of interest, each “interferenced” or scattered EM field (E_{sct}) is detected by a corresponding detecting antenna 48 operating in its detector mode. FIG. 16 is a block diagram of the EM field source-detector cluster 26 of FIG. 10, wherein the cluster is in its detector state. The same reference signal Rrᵢ described in the preceding paragraph is injected into the source-detectors 30 of the EM field cluster 26 (operating in detector mode) immediately downstream from the detecting (receiving) antenna 48. This allows for the R-channel signal Rrᵢ, which is known precisely, to pass through all parts of the detector 30 through which the E_{sct} signal is passed. Therefore, an injection of the R-channel signal into the measuring portions of the source-detectors 30 in both source and detection mode allows for a significant decrease in artifacts caused by temperature and temporary instability of the channel electronics.

The data and other information gathered by the EMT system 10 is provided to the imaging computer 15. The imaging computer 15 carries out a process to solve an inverse problem of electromagnetic field tomography. The solver might be or include, for example, a non-simplified three-dimensional (“3D”) vector solver using Maxwell's equations or a simplified 3D scalar solver or a further simplified 2D scalar solver. As further explained, for example, in U.S. Pat. No. 7,239,731 to Semenov et al., issued Jul. 3, 2007 and entitled “SYSTEM AND METHOD FOR NON-DESTRUCTIVE FUNCTIONAL IMAGING AND MAPPING OF ELECTRICAL EXCITATION OF BIOLOGICAL TISSUES USING ELECTROMAGNETIC FIELD TOMOGRAPHY AND SPECTROSCOPY (the entirety of which is incorporated herein by reference, and which provides background and technical information with regard to the systems and environments of the inventions of the current patent application), such a method may use an iterative procedure based on either a gradient or a Newton calculation approach or it may use a simplified approach using a Born or Rytov approximation. If a non-approximation approach is used it preferably has one or more of the following features, among others: (i) the method is based on minimization of the difference between model scattered fields and measured scattered fields; (ii) the method uses the Tichonov's type of regularization; (iii) one type of the calculation mesh is used in the method; (iv) one step of the iterative procedure is performed as solving of the two sets of direct problems of the same dimension: modeling of the so-called direct wave and modeling of the inverse wave; (v) both the direct wave and the inverse wave are calculated using nonreflecting or metallic boundary conditions; (vi) both the direct wave and the inverse wave are calculated on the same rectangular mesh; (vii) in order to solve the direct problem a conjugate gradient method (“CGM”) might be used; (viii) one step of the CGM uses the sine Fourier transform; (ix) the wave equation for non-uniform media is used to solve the direct problem.

From a mathematical point of view, the methodology utilized in EM field tomography is an inverse problem. It may be formulated in terms of complex dielectric properties ∈ and/or magnetic properties μ and electric and magnetic fields −E, H. The basis is a set of the Maxwell's equations as shown in equation (1) of the aforementioned U.S. Pat. No. 7,239,731, where E and H represent electrical and magnetic fields, respectively, and all other notations are standard.

It is more practical to rewrite these equations in a form of non-uniform wave equations such as that shown in U.S. Pat. No. 7,239,731 equation (2), where

k²=(2π/λ)²∈μ

and λ is a wavelength in vacuum. The EM field tomographic system could be schematically represented as a chamber or other imaging domain with the set of antennae on the surface of the chamber or otherwise on the boundary of the imaging domain. As described previously, some antennae function as EM field sources while the others function as EM field detectors. It is useful to divide electric field E into incident E₀ field and scattered field Eₛ, as shown in U.S. Pat. No. 7,239,731 equation (3) where j is the number of a particular transmitter or source. The equation (2) can be rewritten in the form shown in U.S. Pat. No. 7,239,731 equation (4) where k₀² is a wave number for homogeneous matter and E₀ⱼ is the field produced by the antenna number j.

An object may be described as a distribution of dielectric permittivity and/or magnetic permeability in the imaging domain.

A receiver antenna records the signal, which reflects both incident and scattered fields.

In order to solve equation (4), some boundary conditions may be used on the bound of a calculation domain. Both nonreflecting and reflecting (metallic) boundary conditions may be used on the domain bounds. An interaction of the electromagnetic fields with antennae may be solved as a separate problem. Suitable imaging solvers (Imaging suites) may include, but are not limited to, the Newton, Born, Rytov and MRCSI approaches (in 2D implementations) and the Gradient, Born, Rytov and MRCSI approaches (in 3D implementations). Each of the above approaches can be used separately or in combination with any of the above listed or newly developed approaches. For example, an iterative 3D imaging process might start from a quick 2D Born approach to obtain a first image approximation, and then, starting from this first approximation, a 2D Newton approach may be used for X-number of iterations until a second approximation of image is obtained, and then, starting from the second image approximation, a 3D Gradient method is used for Y-number of iterations to obtain a final imaging results. The exact protocol of the performance of an Imaging Suite is an application case sensitive and is determined by trial method for a particular application (for example breast cancer detection or brain imaging or cardiac imaging etc).

Control Integration System Operation: Imaging-Binding Procedure/Protocol

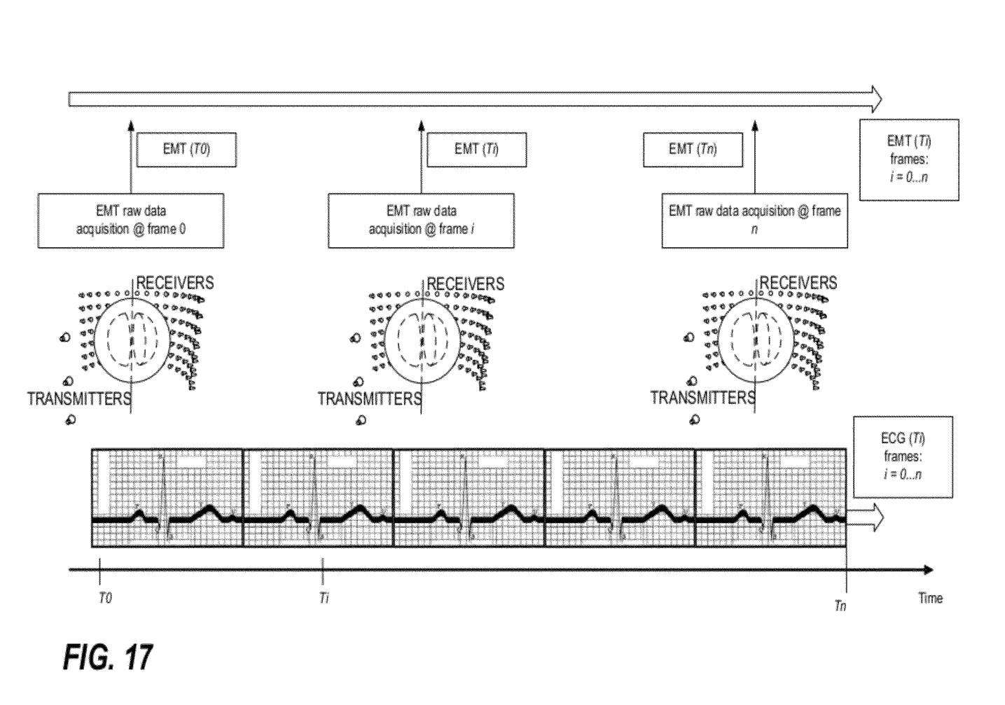

FIG. 17 is a schematic diagram illustrating the acquisition of raw data via both the EMT system 10 and the biomedical electrical recording system 90 of FIG. 8. As shown therein, data is acquired by each system 10,90 at each of a sequence of times Ti, where i=0 . . . n. As shown schematically in FIG. 8, the data from each system 10,90 is then provided to the control integration system 110 for correlation and analysis.

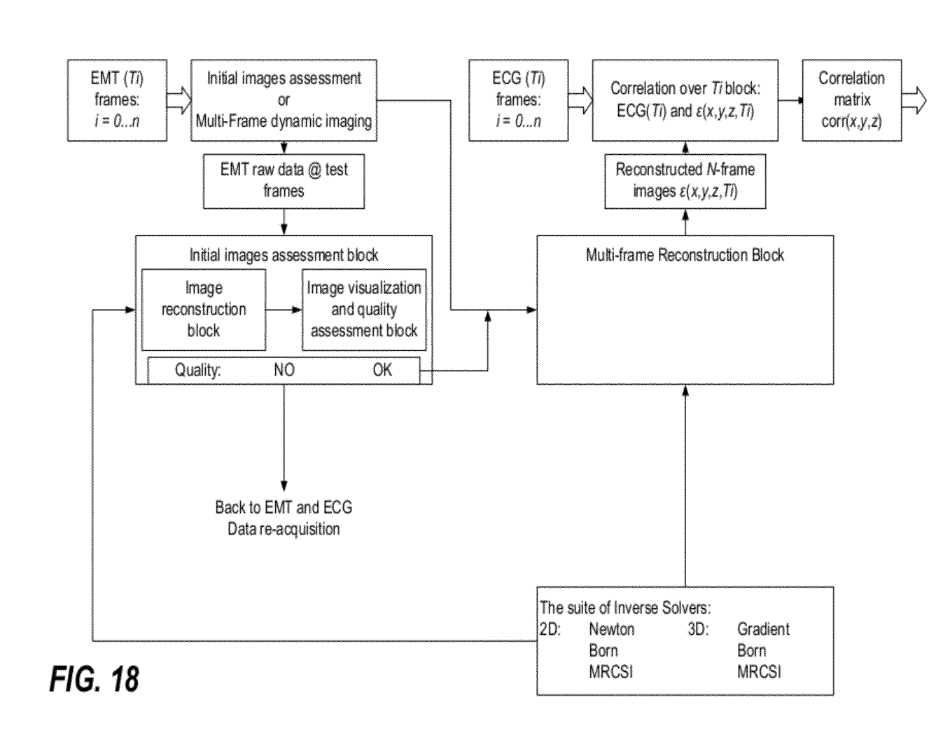

FIG. 18 is a schematic diagram illustrating a process of imaging and determination of correlation of the data from the EMT system 10 with the data from the biomedical electrical recording system 90 as carried out by the control integration system 110, all in accordance with one or more preferred embodiments of the present invention. As shown therein, EMT data (EMT(Ti)) is received from the EMT system 10 and initial images assessment or multi-frame dynamic imaging is conducted. With regard to the former, test frames are periodically selected and assessed, wherein an image is reconstructed and then visualization and quality assessment is applied thereto. Assuming the quality remains acceptable, then the raw data is routed to the multi-frame reconstruction block. On the other hand, if the quality is not acceptable, then data is reacquired as represented in FIG. 17.

After reconstructing multi-frame images (∈(x,y,z,Ti)), the correlation between the matrix ∈(x,y,z,Ti) and ECG (Ti) is obtained over Ti. The output of this part is the matrix of 2D or 3D correlation coefficient corr(x,y,z) between ∈(x,y,z,Ti) and ECG (Ti). The degree of binding is directly proportional to the value of corr(x,y,z). For example, if the correlation coefficient at a given point (xₐ,yₐ,zₐ) has an absolute value closer to 1, this means that nanoparticles at point do, indeed, bind with cells.

It will be appreciated that although FIGS. 17 and 18 illustrate a specific embodiment wherein the biomedical electrical recording system 90 is an ECG system, other biomedical electrical recording systems, some of which were identified hereinabove, may alternatively be utilized, with the primary difference being that instead of recording ECG(Ti) data (when an ECG system is being used), EEG(Ti) data, EMG(Ti) data or EvP(Ti) data is recorded according to the particular type of system 90. Consequentially, the correlation matrix is calculated between EMT(Ti) and the corresponding [E—](Ti) signal.

The suitability of results achieved using the foregoing methods is strongly dependent on the signal of interest from the imaging system 10 being capable of a sufficient digitizing rate. For example, for a typical cardiac rate of 60 beats/minute (i.e., a frequency of 1 Hz, or 1 per 1000 msec) in order to achieve 10 sample points (more would be preferred) each cycle, the digitizing rate should be 100 msec. This means that that the imaging system 10 preferably has an acquisition time of 100 msec or less as well as the ability to accomplish multi-frames data acquisition. Because EMT has been shown to be faster than other imaging technologies, an EMT system 10 is an ideal candidate for such a technology. In one example, one 2D EMT system currently in use has an acquisition time of 13 msec and capable to record 133 frames or more. The in-between frames delay can vary from 15 msec to 1 sec, leading for total acquisition time of 133 frames varying from 2 sec to 22 min (see Semenov S., Kellam J., Sizov Y., Nazarov A., Williams T., Nair B., Pavlovsky A., Posukh V., Quinn M. “Microwave tomography of extremities: 1) Dedicated 2D system and physiological signatures,” Phys. Med. Biol., 56 (2011) 2005-2017; Semenov S., Kellam J., Nair B., Sizov Y., Nazarov A., Williams T., Nair B., Pavlovsky A., Quinn M. “Microwave tomography of extremities: 2) Functional fused imaging of flow reduction and simulated compartment syndrome,” Phys. Med. Biol., 56 (2011) 2019-2030). In another example, one 3D EMT system currently under construction will reportedly have a 3D acquisition time of 20-50 msec per frame and will be able to record up to 250 consequential frames.

Notably, it may also be possible to use the methods described herein to assess the binding efficiency of nanoparticles with malignant cells. There are recent reports demonstrating the differences in membrane potentials in normal and malignant cells (see A. A. Marino, I. G. Iliev, M. A. Schwalke, A. Gonzalez, K. C. Marler and C. A. Flanagan, “Association between cell membrane potential and breast cancer,” Tumor Biol, 15: 82-89, 1994).

### Enhanced Imaging Capabilities

In addition to the assessment capabilities described above, the methods and system of the present invention, in at least some embodiments, have enhanced imaging capabilities deriving from the use of EMT with nanoparticles composed of ferroelectric (Fel) and either magnetic (Mag), metal (e.g., gold) (Met), or both or all of them (i.e., Fel-Mag, Fel-Met or Fel-Mag-Met)

What is reconstructed by EMT is

k˜∈·μ=(∈′+j∈″)*(μ′+jμ″)

In traditional EMT, it is assumed that μ=1+j0, which is a very reasonable assumption for most biological tissues (except for example for erythrocytes at microscopic level). However, if it is assumed that a “reasonable” concentration of nanoparticles is achieved within a target volume of tissue comparable with the wavelength of EM radiation in the tissue, then it follows that within that volume there are complex, non-zero ∈ and μ. Since the overall dielectric properties ∈ are a combination of the dielectric properties of the tissue and those of the nanoparticles, while the magnetic properties μ are only from the nanoparticles, then within this volume:

k˜∈·μ=(∈′+j∈″)*(μ′+jμ″)=(∈′μ′−∈″μ″)+j(∈″μ′+∈′μ″)

This difference is important. In particular, a very important component is the “effective” real part of

k=(∈′μ′−∈″μ″)

which can have a negative value within a volume of interest, thereby implying a contraction in EMT. In view of this, different effects may be achieved using different nanoparticle materials, alone or in composite nanoparticles. More particularly, a high ∈* might be achieved by using ferroelectric nanoparticles; a high μ* might be achieved by using magnetic nanoparticles; and a significant local rise in conductivity σ[S/m] might be produced by using gold and certain other metal nanoparticles. For example, the typical conductivity of biological tissues within MHz-GHz frequencies does not exceed single digits of [S/m], while the same one for metal (copper) is about 5.9×10⁷ [S/m]. The latter contrast in conductivity is also beneficial in the context of therapeutics protocols, described later. Notably, these effects may be produced either in combination or independently by using composite nanoparticles or nanoparticles of individual materials.

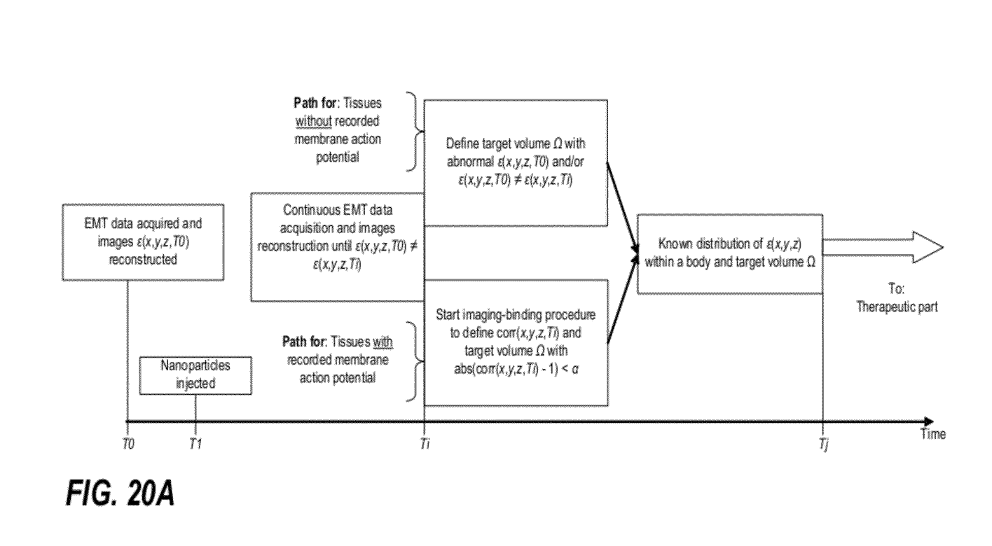

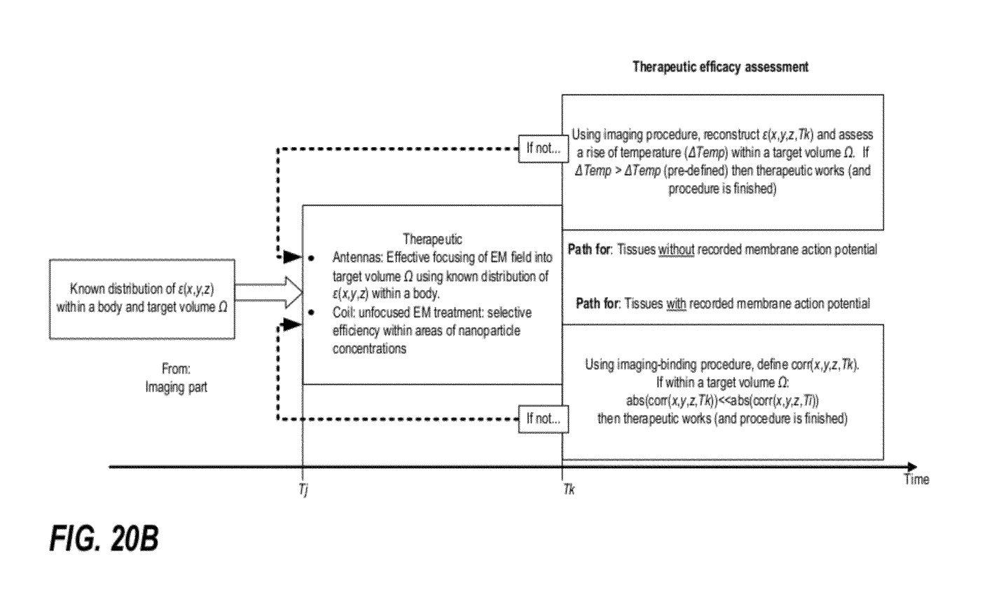

A protocol for imaging is illustrated in FIG. 20A. Such a protocol further discussed in a subsequent section.

### Therapeutic Procedures

In addition to providing enhanced imaging capabilities, the methods and system described herein may be incorporated into one or more therapeutic procedures. By way of background, it will be appreciated that short power electrical pulses (E-pulses) may be utilized to disrupt (or open pores in) both the cellular membrane of targeted cells (for example, malignant cells) and of the nanoparticle shell in order to release a drug (for example, an anticancer drug) from composite nanoparticles. One or both of an electrical mechanism (E-mechanism) and a thermogenic mechanism (T-mechanism) of membrane disruption and tissue treatment may be used. The methods described herein are more targeted and precise than traditional hyperthermia methods mediated by one or the other of magnetic or ferroelectric nanoparticles on their own.

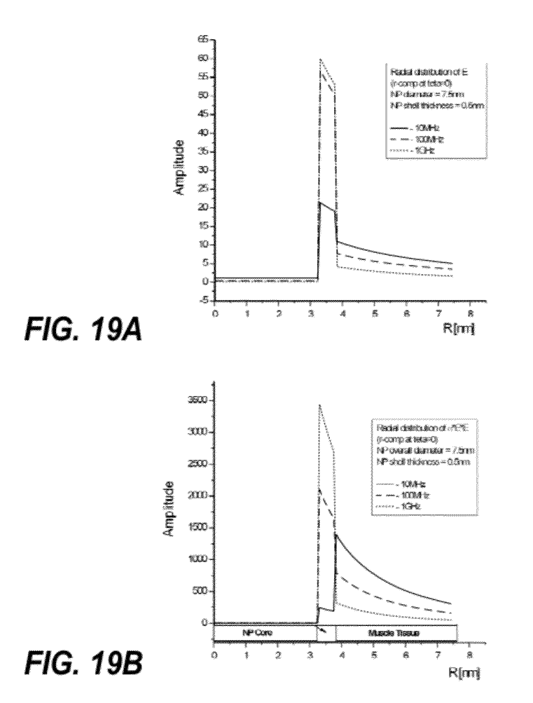

FIGS. 19A and 19B are graphical illustrations of computer simulation results of the radial distribution of electric field E and thermogenic factor (σ*E*E), at various frequencies, in and across the boundary of an exemplary nanoparticle. The nanoparticle has a radius of about 3.75 nm (diameter of about 7.5 nm) and a shell thickness of about 0.5 nm. A high increase in both E and T (the latter being mediated through an increase in SAR) in the shell of the nanoparticle can be seen. Since the thermogenic factor (σ*E*E) is proportional to local conductivity σ, it may be beneficial to use a composite (with a metal component) or a single metal-component nanoparticles with high conductivity, as described above.

The treatment (therapeutic) procedure is based on the use of different methods (mechanisms) or their combinations: E-mechanism, T-mechanism, combined ET or TE. In at least some embodiments, any of the foregoing may be combined with drug release from nanoparticles with further on-line monitoring of the results of treatment. On-line monitoring may be based on the use of the EMT methodology with the information about an efficacy of therapeutics obtained from two imaging protocols: i) the imaging-binding protocol described previously (since at point (x,y,z) the number of nanoparticles that are bound is expected to be decreased as cell-nanoparticle complexes are disrupted) and ii) a classical dielectric properties imaging protocol (since dielectric properties are dependent on temperature, a rise in the localized temperature at any point (x,y,z) will result in a corresponding change in dielectric properties).

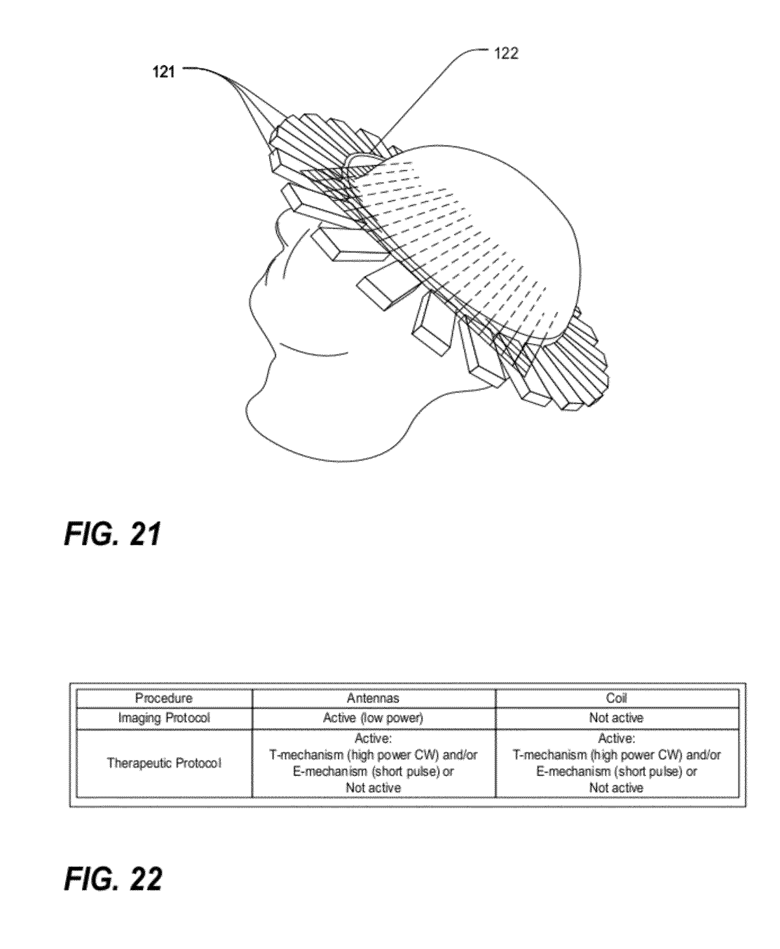

FIGS. 20A and 20B are schematic diagrams illustrating the use of an EMT imaging protocol in conjunction with a therapeutic protocol in accordance with one or more preferred embodiments of the present invention, while FIG. 21 is an illustrative computer-simulated human head model and experimental setting for use in illustrating the protocols of FIGS. 20A and 20B. As representatively illustrated in FIG. 21, two components, an array of antennas 121 and a coil 122, are utilized in treatment involving the head of a human patient. It will be appreciated that although only a single set of antennas 121 and one coil 122 are shown, multiple antenna sets and coils could alternatively be utilized to effectuate more comprehensive operation.

FIG. 22 is a tabular representation of the functionality carried out by the antennas 121 and coil 122 during the imaging protocol and the therapeutic protocol of FIGS. 20A and 20B, respectively. As shown therein, only the antennas 121 are active (at low power) during the imaging protocol, while during the therapeutic protocol both the antennas 121 and the coil 122 may be active. The antennas 121 and coil 122 may each use a T-mechanism (high power CW), an E-mechanism (short pulse), both, or neither.

As shown in FIG. 20A, EMT data is acquired and images (∈(x,y,z,T0)) reconstructed beginning at time=T0. At time=T1, composite nanoparticles are injected into the subject, and EMT data acquisition and image reconstruction occurs at each time Ti until ∈(x,y,z,Ti) does not equal ∈(x,y,z,T0). At this point, for tissues without recorded membrane action potential, the target volume Ω is defined with abnormal ∈(x,y,z,T0) and/or ∈(x,y,z,Ti) does not equal ∈(x,y,z,T0). Alternatively, for tissues that do have recorded membrane action potential, the imaging-binding procedure is started in order to define corr(x,y,z,Ti) and the target volume Ω with abs(corr(x,y,z,Ti)−1)<α. A known distribution of ∈(x,y,z) may then be developed within a body and target volume Ω, with completion at time=Tj.

As shown in FIG. 20B, the therapeutic protocol involves the use of the antennas 121, the coil 122, or both. The antennas 121 may be used for effective focusing of an EM field into the target volume Ω using the known distribution of ∈(x,y,z) within the body. The coil 122 may be used for unfocused EM treatment, with selective efficiency within areas of nanoparticle concentrations. This continues until time=Tk, at which point a therapeutic efficacy assessment may occur. For tissues that did not have recorded membrane action potential, an imaging procedure may be used to reconstruct ∈(x,y,z,Tk) and the rise of temperature (ΔTemp) may be assessed within the target volume Ω. If the change in temperature is greater than some predefined amount, then the therapy may be considered to be successful, and the procedure may be terminated. If not, then control returns to the therapeutic step described previously. On the other hand, for tissues that had recorded membrane action potential, an imaging-binding procedure may be used to define corr(x,y,z,Tk). If, within the target volume Ω, abs(corr(x,y,z,Tk)) is substantially less than abs(corr(x,y,z,Ti), then the therapy may be considered to be successful, and the procedure may be terminated. If not, then control returns to the therapeutic step described previously.

In some embodiments, composite nanoparticles are used for the imaging and therapy, wherein the steps of imaging the biological tissue and implementing a therapy may be based on two different materials therein. In this case, the imaging step may be based on a first material (such as a ferroelectric material) in the composite nanoparticles and the therapy may be based on a second material. In other embodiments, non-composite nanoparticles are used for the imaging and therapy, with both steps being implemented using the same material in the nanoparticles.

One or more methods and systems of the present invention have many advantages. As was stated previously, knowledge of the distribution of dielectric properties of tissues within a body and of the distribution of nanoparticles within the body in combination with knowledge of an EM field pattern within the body would be very useful to the success of EM hyperthermia. Thus, an imaging modality for obtaining such knowledge directly would facilitate fast, on-line imaging of time-varying tissue properties even during an EM hyperthermia procedure. Since with EMT an imaging is based on differences in tissue dielectric properties and concentrated composite nanoparticles present a dielectric contrast, EMT is an imaging modality which is able to provide direct information about distribution of dielectric properties of tissues within a body and, consequentially, the EM field distribution and distribution of nanoparticles.

Based on the foregoing information, it will be readily understood by those persons skilled in the art that the present invention is susceptible of broad utility and application. Many embodiments and adaptations of the present invention other than those specifically described herein, as well as many variations, modifications, and equivalent arrangements, will be apparent from or reasonably suggested by the present invention and the foregoing descriptions thereof, without departing from the substance or scope of the present invention.

Accordingly, while the present invention has been described herein in detail in relation to one or more preferred embodiments, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements; the present invention being limited only by the claims appended hereto and the equivalents thereof.

1.-47. (canceled)

48. A system for electromagnetic imaging and therapeutics using composite nanoparticles, comprising:
(a) an electromagnetic tomography system adapted to image a biological tissue via a material in the composite nanoparticles;
(b) a therapeutic application system adapted to implement a therapy, via a material in the composite nanoparticles, at least partly on the basis of information received from the electromagnetic tomography system; and
(c) a control system adapted to assess an effect of the therapy and to control further implementation of the therapy based on the assessment.

49. The system of claim 48, wherein the electromagnetic tomography system is adapted to image the biological tissue via a first material in the composite nanoparticles, and wherein the therapeutic application system is adapted to implement the therapy via a second material in the composite nanoparticles.

50. The system of claim 49, wherein the first material in the composite nanoparticles includes a ferroelectric material.

51. The system of claim 49, wherein the second material in the composite nanoparticles includes a magnetic material.

52. The system of claim 49, wherein the therapy utilizes an electrical mechanism.

53. The system of claim 49, wherein the therapy utilizes a thermogenic mechanism.

54. The system of claim 49, wherein the therapeutic application system includes antennas disposed at points around the biological tissue.

55. The system of claim 49, wherein the therapeutic application system includes one or more coil arranged around the biological tissue.

56. The system of claim 48, wherein the nanoparticle material via which the electromagnetic tomography system is adapted to image the biological tissue is the same nanoparticle material via which the therapeutic application system is adapted to implement the therapy.

57. A method for electromagnetic imaging and therapeutics using composite nanoparticles, comprising:
(a) imaging a biological tissue, via an electromagnetic tomography system, using a material in the composite nanoparticles;
(b) implementing a therapy, via a therapeutic application system, using a material in the composite nanoparticles, wherein the implementation is carried out at least partly on the basis of information received from the electromagnetic tomography system;
(c) assessing an effect of the therapy; and
(d) controlling further implementation of the therapy based on the assessment.

58. The system of claim 57, wherein the step of imaging the biological tissue uses a first material in the composite nanoparticles, and wherein the step of implementing the therapy uses a second material in the composite nanoparticles.

59. The method of claim 58, wherein the first material in the composite nanoparticles includes a ferroelectric material.

60. The method of claim 58, wherein the second material in the composite nanoparticles includes a magnetic material.

61. The method of claim 58, wherein implementing the therapy includes utilizing an electrical mechanism.

62. The method of claim 58, wherein implementing the therapy includes utilizing a thermogenic mechanism.

63. The method of claim 58, wherein implementing the therapy includes disrupting or opening pores in the cellular membrane of targeted cells.

64. The method of claim 58, wherein implementing the therapy includes disrupting or opening pores in the shell of the nanoparticles to release a drug therefrom.

65. The method of claim 57, wherein the step of imaging the biological tissue and the step of implementing the therapy use the same material in the composite nanoparticles.
